(19) United States
(12) Patent Application Publication
(10) Pub. No.: US 2023/0284645 A1
(43) Pub. Date: Sep. 14, 2023

(54) INFUSION OF CANNABIDIOL (CBD) IN BULK GREEN COFFEE BEANS AND INTEGRATION INTO COFFEE PRODUCTION

(71) Applicant: Simon Gonzales, Parker, CO (US)

(72) Inventor: Simon Gonzales, Parker, CO (US)

(73) Assignee: Columbia Zona, Parker, CO (US)

(21) Appl. No.: 17/690,062

(22) Filed: Mar. 9, 2022

(51) Int. Cl. A23F 5/02; A23L 33/10; A61K 31/352

(52) U.S. Cl. CPC A23F 5/02; A23L 33/10; A61K 31/352

(57) ABSTRACT

Described is a process to manufacture CBD coffee through infusion of CBD into the green coffee bean.

### FIELD OF THE INVENTION

The present disclosure relates to a method and use of infusion of organic cannabidiols (CBDs) into green coffee beans and integration into the roasting and the grinding process. The disclosure further provides for CBD-infused coffee beans for human consumption.

### BACKGROUND

The following description provides a summary of information relevant to the present disclosure and is not an admission that any of the information provided or publications referenced herein is prior art to the present disclosure.

Coffee statistics from the International Coffee Organization (ICO), states 1.4 billion cups of coffee consumed a day worldwide with forty-five percent consumed in the United States alone.

Cannabidiols (CBDs) have been used previously in the coffee industry by spraying CBDs on already roasted coffee or using chemical solvents, thus altering the flavor and aroma of the final product, factors of great importance in the coffee industry.

The current state of processing with regard to CBD and coffee is applying a CBD spray post-roasting of the coffee, which is a surface process. The CBD left only on the green bean surface prior to roasting, results in the CBD material being volatilized due to the temperature of the roasting process. Other known processes developed for the infusion of green beans use chemical solvents which can leave harmful residuals for human consumption.

The present disclosure teaches a method for production wherein CBD is infused into green coffee beans, to thereafter being roasted and grinded, resulting in a final coffee product to be consumed comprising CBD that is bioavailable to humans. The resultant coffee comprises the appropriate flavor and odor for coffee consumption. The process gives the opportunity to roasters and distributors to apply their open knowledge to develop a final product ensuring premium quality coffee and delivery of CBD.

### SUMMARY

The present disclosure provides a method for infusion of the bulk (body or matrix of the bean) of green coffee beans with CBDs comprising a method to open the pores of the green coffee bean with heat exchange and allow a solution of CBD, with a controlled temperature (within given parameter of the process), to penetrate the bean through an osmotic process, wherein the pores of the bean are filled with CBD, wherein the porosity is physiologically found in the body of the coffee bean, and wherein the penetration of the CBD into pores of the green coffee bean increases CBD retention into the coffee bean. In one embodiment, the heat exchange is steam or heated water. In one embodiment, pre-heating of the bean prior to the steam treatment is applied to lower surface and internal water contents of the green bean for repeatability of the process. In one embodiment, a green coffee bean is infused with a quantifiable amount of CBD. In one embodiment, the dose of CBD in the resultant coffee is determinable. In one embodiment, the dose of CBD is determinable in the green coffee bean.

In one embodiment, a method is provided for an infusion of CBD in the bulk of the green bean using a pore opening method with steam or heated water. Once the pores have opened, a temperature-controlled CBD is sprayed on the green beans or green beans are submerged in a CBD solution. CBD infusion is attained with an oil or water-soluble base material/liquid for the process. To improve the infusion process, in one embodiment, surfactants are added to alter the hydrophobic or hydrophilic nature of the bean contents. In one embodiment, a pre-heat of the bean prior to the steam treatment is applied to lower surface and internal water contents of the green bean for repeatability of the process.

In one embodiment, a therapeutic effect (e.g., treating, preventing or ameliorating pain) may be achieved by administering coffee product produced from the CBD infused green coffee bean.

### BRIEF DESCRIPTION OF THE DRAWINGS

FIG. 1 illustrates the infusion process of green coffee beans with CBD using steam or hot water, followed by the application of CBD as a liquid spray or by submersion in CBD liquid.

FIG. 2 illustrates a method of mixing green coffee beans infused with CBD with uninfused green coffee beans, followed by the roasting and grinding process.

FIG. 3 illustrates an alternate method of mixing roasted coffee beans infused with CBD with roasted uninfused coffee beans, followed by roasting then grinding or grinding then roasting.

FIGS. 4A-4B illustrate the steam module used to treat green coffee beans prior to infusion with CBD. FIG. 4A shows the attribute and function of the components in the steam module. FIG. 4B shows the configuration of the steam module.

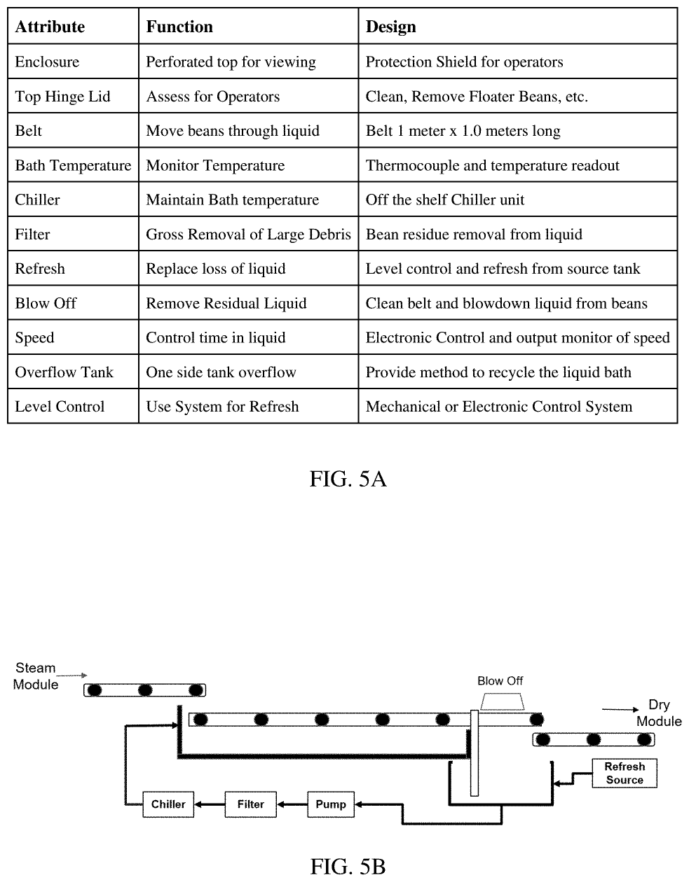

FIGS. 5A-5B illustrate the receiving module used to infuse beans from the steam chamber with CBD. FIG. 5A shows the attribute and function of the components in the receiving module. FIG. 5B shows the configuration of the receiving module.

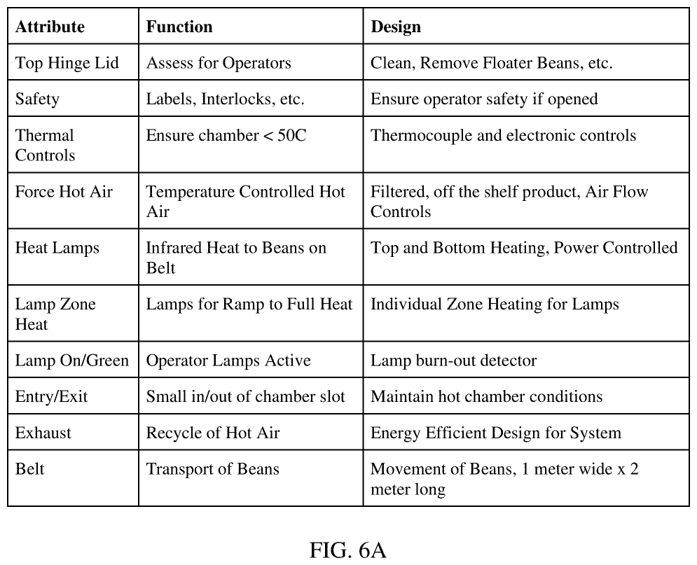

FIGS. 6A-6B illustrate the drying module used to reduce water content of the beans after infusion. FIG. 6A shows the attribute and function of the components in the drying module. FIG. 6B shows the configuration of the drying module.

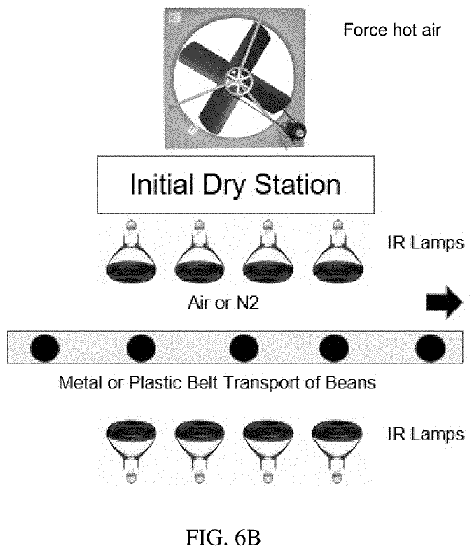

### DETAILED DESCRIPTION

Reference will now be made in detail to representative embodiments of the invention. While the invention will be described in conjunction with the enumerated embodiments, it will be understood that the invention is not intended to be limited to those embodiments. On the contrary, the invention is intended to cover all alternatives, modifications, and equivalents that may be included within the scope of the present invention as defined by the claims.

One skilled in the art will recognize many methods and materials similar or equivalent to those described herein, which could be used in and are within the scope of the practice of the present invention. The present invention is in no way limited to the methods and materials described.

Unless defined otherwise, technical and scientific terms used herein have the same meaning as commonly understood by one of ordinary skill in the art(s) to which this invention belongs. Although any methods, devices, and materials similar or equivalent to those described herein can be used in the practice or testing of the invention, the preferred methods, devices and materials are now described.

All publications, published patent documents, and patent applications cited in this disclosure are indicative of the level of skill in the art(s) to which the disclosure pertains. All publications, published patent documents, and patent applications cited herein are hereby incorporated by reference to the same extent as though each individual publication, published patent document, or patent application was specifically and individually indicated as being incorporated by reference.

As used in this disclosure, including the appended claims, the singular forms “a,” “an,” and “the” include plural references, unless the content clearly dictates otherwise, and are used interchangeably with “at least one” and “one or more.” Thus, reference to “an aptamer” includes mixtures of aptamers, and the like.

As used herein, the term “about” represents an insignificant modification or variation of the numerical value such that the basic function of the item to which the numerical value relates is unchanged.

The term “each” when used herein to refer to a plurality of items is intended to refer to at least two of the items. It need not require that all of the items forming the plurality satisfy an associated additional limitation.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but may include other elements not expressly listed or inherent to such process, method, product-by-process, or composition of matter.

As used herein, the term “bulk” refers to. body or the matrix of the green coffee bean.

As used herein, the term “cannabinoid”, “CBD” or “cannabinoids”, “CBDs” in this disclosure refers to any of the diverse chemical compounds that act on cannabinoid receptors on human cells. They include the phytocannabinoids found in cannabis, hemp seed oil, other plants, and synthetic cannabinoids manufactured artificially. They include the phytocannabinoids delta-9-tetrahydrocannabinol (THC), cannabidiol (CBD), cannabinol (CBN) cannabigerol (CBG), cannabigerol (CBG), cannabichromene (CBC), cannabicyclol (CBL), canabivarol (CBV), tetrahydrocannabivarin (THCV), cannabidivarin (CBDV), cannabichromevarin (CBCV), cannabigerol monoethyl ether (CBGM), or the like; or mixtures or combinations thereof. Other botanical cannabimimetics include N-alkylamides from Echinacea and B-caryophyllene. They include mixtures of phytocannabinoids separated from the plant by extraction techniques and high purity cannabinoids obtained by purification from natural sources or via synthesis.

As used herein, “CBD coffee” refers to coffee for drinking, wherein CBD has been infused, and the coffee possess a therapeutic effect due to the infusion of CBD.

As used herein, “Non-CBD coffee” refers to coffee for drinking, wherein CBD has not been infused, and the coffee does not possess a therapeutic effect due to CBD.

As used herein, “bioavailability” refers to the physiological availability of a given amount of a drug as distinct from its chemical potency; proportion of the administered dose that is absorbed into the bloodstream.

As used herein, “green bean,” refers to the coffee bean prior to roasting and grinding

As used herein, “temperature control,” refers to process parameters for the method of green bean infusion.

As used herein, the term “therapeutically effective amount” generally means the amount necessary to ameliorate at least one symptom of a disorder or condition to be prevented, reduced, or treated as described herein. It is emphasized that a therapeutically effective amount of CBD that is administered to a particular individual in a particular instance will not always be effective in treating the conditions/diseases described herein, even though such dosage is deemed to be a therapeutically effective amount by those of skill in the art.

Cannabinoids are molecules found in plants and humans. Those found in plants are known as phytocannabinoids and those found in humans are known as endocannabinoids. There have been described more than 80 different chemicals in this plant. The most well-known phytocannabinoids molecules produced by the Cannabis sativa plant are the trans-Δ9-tetrahydrocannabinol (THC) and the Cannabidiol (CBD).

Cannabinoids molecules interact with the endocannabinoid system in humans which can be used to treat different health issues such as anxiety, joint pain, muscle pains, Parkinson and Crohn diseases and many other conditions. Since CBD has been studied as a new drug, it is legal to include it in the food, diet supplements industry and beauty products, only if it contains less than 0.3% of THC. CBD cannot be included in the products commercialized with therapeutic purposes.

People around the globe suffering from conditions mentioned above benefit from the activation of the endocannabinoid receptors by consuming CBD, hence improving their life quality. Nevertheless, health professionals have expressed the difficulty of patients to intake pills, drops or any other type of medicine, by either refusing, forgetting or just disregarding advice of taking the medications on a daily basis.

Adding supplements or nutritional value to food products has been done for decades, with the goal to reach more people and improve the community intake of nutrients and provide health benefits. This type of food is known as “functional foods”. Functional food ingredients that have been consumed for a long time do not require testing by the FDA in the U.S. since they are Generally Recognized as Safe (GRAS). Cannabis products, including hemp, have been consumed as food for many years and considered safe under this recognition.

Cannabidiols (CBDs) have been used previously in the coffee industry by spraying CBDs on already roasted coffee or using chemical solvents, thus altering the flavor and aroma of the final product, factors of great importance in the industry. The present disclosure teaches infusion of the bulk of green coffee beans with CBD by developing a method to open up the pores of the bean with heat exchange (steam or heated water) and allow the solution of CBD, with a controlled temperature, to penetrate the bean naturally by an osmotic process, filling up spaces physiologically found in the body of the coffee bean and increasing CBD retention. A pre-heat of the bean prior to the steam treatment is applied to lower surface and internal water contents of the green bean for repeatability of the process. This process gives the opportunity to roasters and distributors to apply their open knowledge to develop a final product ensuring premium quality coffee and delivery of CBD.

Dilution of the CBD is attained with deionized or mineralized water depending on the requirements of the product taste, flavor, or aroma. In addition, control of the mass transport for total CBD infusion into the bulk of the bean. CBD material/liquid temperature variation due to thermal energy added from the steamed beans is algorithmic adjusted with chiller equipment and controlled with thermocouple measurement.

Blending methods of the CBD bulk coffee is achieved with mixing of Non-CBD coffee with volume percent by the desired final product results for flavor, aroma, roast, and CBD concentration. Blending green beans of CBD and Non-CBD prior to roasting or after roasting to create a homogenous mixture is applicable to adjust aroma, flavor, and CBD concentration from base material infused. A non-blended process is achieved with the CBD green beans roasted and grinded with no subsequent mixing with Non-CBD green or roasted beans. A dry method of beans consisted of radiative, conductive, and convective heating to remove bean internal and surface water contents. Nitrogen for dry method to improve removal of surface and internal water contents along with bean heating exposure. Ultraviolet (UV-C) applied to the bean process for sterilization and improving food quality levels of the product.

A method to measure/detect the CBD concentration obtained with High Performance Liquid Chromatography (HPLC) or other means to detect concentration levels.

### EXAMPLES

The following examples are provided for illustrative purposes only and are not intended to limit the scope of the invention as defined by the appended claims. All examples described herein were carried out using standard techniques, which are well known and routine to those of skill in the art.

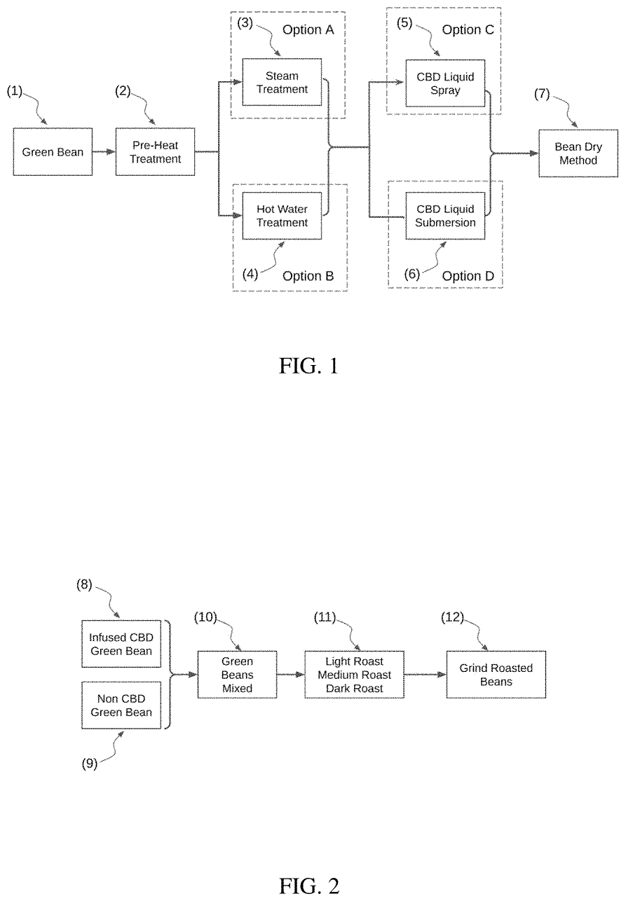

The process (FIG. 1) is initiated with a green bean (1) as the source raw material. Due to the nature of green bean upstream processing, variation of water content is normal for incoming bean material. A pre-measure of the water content (AgraTronix Portable Coffee Bean Moisture Tester Model 08150 or other apparatus) is applicable to define baseline conditions. The pre-heat (2) time and temperature is used for an algorithmic adjustment of bean water contents prior to the heated water step (3, 4).

Two (FIG. 1) methods are applicable to open up the bean pores including Option A steam (3) or Option B hot (4) water treatment. Such water at this step, may either be mineralized or deionized with dependence on the impact on flavor, aroma or mass transport of the CBD infusion.

Movement of the treated green beans from steam (3) and hot water (4) step and temperature maintained with ambient heat or infrared lamps. The CBD infusion (FIG. 1), includes Option C spray (5) or Option D submersion (6). Parameters to control the CBD infusion include, dwell time liquid exposure of the bean, liquid temperature, liquid dilution, and contact exposure of total bean surface area.

CBD spray method (3) may occur from a single source or multiple outlets of dispersion. In addition, top and bottom spray (3) to improve coverage of beans for infusion. The method to submerge beans for infusion with a consideration green beans do not float (density greater than CBD solution and water) in the CBD liquid. The liquid from both the spray and submersion is recycled with collection to be pumped, filtered, and temperature adjusted prior to reuse including spiking to adjust the CBD concentration for infusion process repeatability and reproducibility.

CBD used in the process (FIG. 1) from a water-soluble source or oil-base. Both available in multiple formulations with adjustments of concentration and viscosity as needed for processing through the spray (5) or submersion (6) method.

Post CBD liquid infusion a drying step (7) is used to reduce the overall water contents of the bean and reduce external surface wetness for further processing. Such drying based on temperature, time, and a source of convective, conductive, or radiative or a combination of such methods to dry the bean to conditions of water percent prior to roasting.

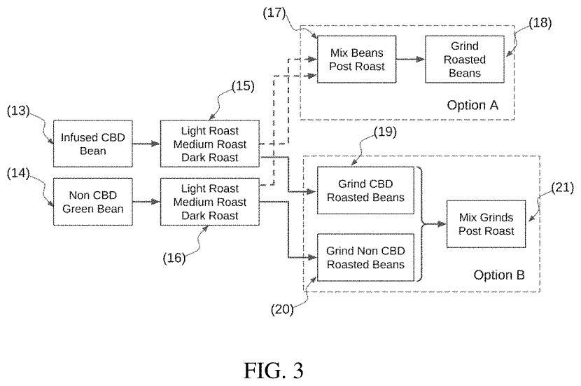

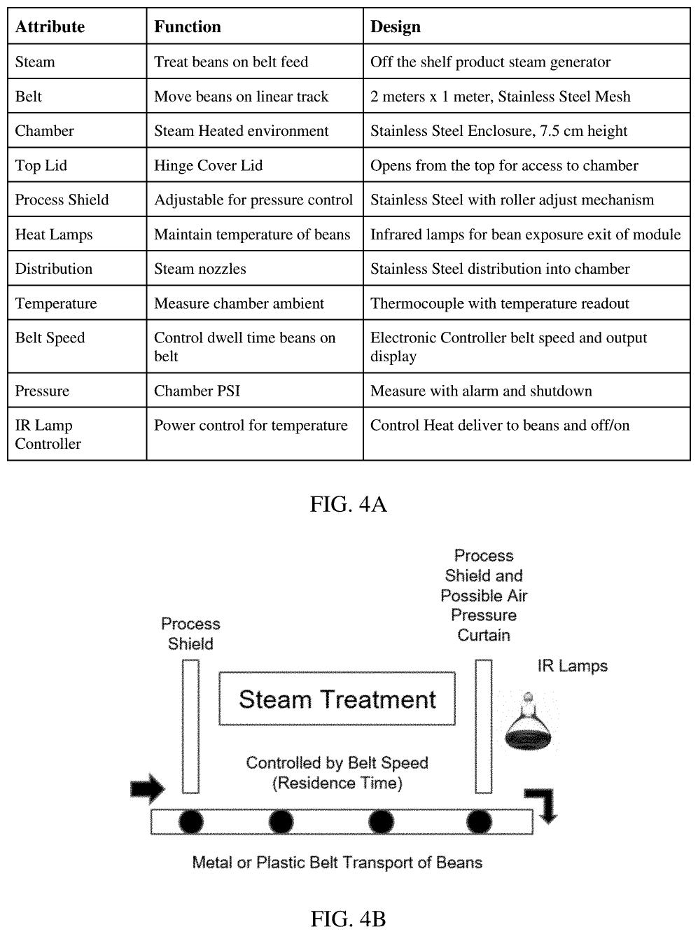

In order to have a commercial product from the infusion process, methods to blend and roast are required (FIG. 2 and FIG. 3). The processes defined are designed to provide options for the end user from the base material of infused green beans. The disclosure provides infinite blending and adjustment of the CBD concentration along with coffee aroma and flavor, as would be known by one of skill in the art.

FIG. 2 Infusion to Blending and Roast Process, provides the method to mix green beans infused with CBD (8) and beans with no CBD (9). Once the beans are mixed (10), the preferred roast (11) type of light, medium, or dark may be completed depending on the desired output of the final blend. The final step post roast is the grind (12) completes the coffee process and the blend of CBD and Non-CBD coffee as a homogeneous mixture. This method described in FIG. 2 may eliminate the Non-CBD coffee (9) for CBD (8) processed through roast (11) and grind (12). As defined by this process flow the nature of the coffee business requires adjustment to aroma and flavor, with CBD concentration per unit volume being allowed with this method to integrate the invention with process manufacturing.

FIG. 3 represents an alternate method to FIG. 2 that moves the blending to a secondary state of individual CBD (13) and Non CBD Coffee (14) being roasted individually (15, 16), and then processed through option A or option B. In option A, the infused coffee beans (13) post roast (15) are mixed (17) with the Non-CBD beans (14) post roast (16), and then grinded (18) to provide a homogeneous blend. Option B, the CBD coffee post roast (15) and Non-CBD coffee (16) are individually grinded (19, 20) and then mixed grinds (21) to create the homogenous blend. Both option A and B provide the control of concentration of CBD in the final blend, and adjustment of aroma and flavor with an infinite blending process for the end user requirements.

What is claimed is:

1. A method of using non solvent or chemical base processes to open pores or existing pores of a green coffee bean with subsequent infusion of cannabidiol (CBD) into the bulk of the bean.

2. A method of preheating a green coffee bean where the water content of the bean is adjusted for the infusion process to control repeatability and reproducibility.

3. A method using the beans natural pore structures to expand the size using steam or hot water as a means to introduce CBD.

4. The method of claim 1, where the infusion can be enhanced by a surfactant.

5. A method where the use of steam condensate with residual caffeine and flavor is applied as a reabsorption material or as an additional infusion of CBD after the submersion or spray process.

6. A method where the CBD liquid used for infusion is water-based.

7. A method where the CBD liquid used for infusion is oil-based.

8. The method of claim 6 or 7 where the concentration of the CBD liquid is adjusted for the infusion process and associated equipment by dilution.

9. The method of claim 6 or 7 where the viscosity of the CBD liquid is adjusted for the infusion process and associated equipment by dilution.

10. A method of controlling the CBD concentration with periodic addition of additional solutions or CBD components.

11. A method wherein a CBD liquid tank comprising a nitrogen (N2) blanket is used to reduce CO2 and other ambient incursions or changes to the base materials.

12. A method where a CBD liquid tank is used with a single side overflow to allow reprocessing of material and removal of floating green beans.

13. A CBD coffee product, wherein the product is made by the process of infusing green coffee beans with CBD product comprising using non solvent or chemical base processes to open the pores of a green coffee bean with subsequent mass transport of CBD solutions into the bulk of the bean.

14. A method for producing a coffee product comprising using non solvent or chemical base processes to open the pores of a green coffee bean with subsequent mass transport of CBD solutions into the bulk of the bean.
